(19) United States
(12) Patent Application Publication
(10) Pub. No.: US 2013/0096070 A1
(43) Pub. Date: Apr. 18, 2013

(54) PEPTIDE TARGETING OF INNER EAR CELLS

(71) Applicants: Hinrich Staecker, Leawood, KS (US); Peter Gochee, Roeland Park, MO (US)

(72) Inventors: Hinrich Staecker, Leawood, KS (US); Peter Gochee, Roeland Park, MO (US)

(73) Assignee: UNIVERSITY OF KANSAS, Lawrence, KS (US)

(21) Appl. No.: 13/263,719

(22) PCT Filed: Apr. 6, 2010
(86) PCT No.: PCT/US2010/030062

§ 371 Date: Apr. 19, 2012

Related U.S. Application Data
(60) Provisional application No. 61/167,314, filed on Apr. 7, 2009.

(51) Int. Cl. A61K 38/08; C07K 7/06

(52) U.S. Cl. CPC A61K 38/08; C07K 7/06
USPC 514/20.9; 530/329; 435/69.1; 514/21.7; 422/261

(57) ABSTRACT

Peptide targeting moieties that target the cells of the inner ear can be used for targeted therapeutics. As such, nucleic acids and/or drugs can be associated with the targeting moieties in order to provide therapeutics that are delivered to specific cells in the inner ear. Conjugation of drugs or gene therapy vectors to cell specific peptides may allow the treatment of individual cell types within the inner ear. The peptide targeting moieties can be polypeptides having the sequences of Table 1 in an unnatural configuration. The polynucleotide can either consist of the sequence or include additional polypeptides attached to the ends of the sequences shown in Table 1. The polynucleotide can be in a non-native configuration. For example, the polypeptide is selected from the following: a-h-p-h-h-s-m (SEQ ID NO: 12); h-p-h-h-r-i-f (SEQ ID NO: 29); t-v-p-q-l-t-t (SEQ ID NO: 1); s-t-t-k-l-a-l (SEQ ID NO: 2); m-e-g-y-i-h-r (SEQ ID NO: 3); h-a-i-y-p-r-h (SEQ ID NO: 5); h-s-r-l-l-d-q (SEQ ID NO: 6); i-q-s-p-h-f-f (SEQ ID NO: 7); or y-a-a-h-r-s-h (SEQ ID NO: 8).

### CROSS-REFERENCE

This application claims the benefit of U.S. Provisional Application No. 61/167,314, filed Apr. 7, 2009, which provisional application is incorporated herein by specific reference in its entirety.

### BACKGROUND

Sensorineural hearing loss and vestibular dysfunction represent a collection of inner ear disorders for which limited surgical and medical treatments exist. Gene therapy may lead to additional therapeutic options for individuals affected with these disorders. Recent research has shown auditory hair cell replacement, prevention of hair cell degeneration, and improvement in hearing. Several vectors have been used for gene delivery, including adenovirus, adeno-associated virus, and herpes simplex virus.

Delivery of genes via these vectors is dependent on the interaction of the vector coat with receptors on a cell. Therefore, cells lacking receptors for the vector binding protein will not be targeted. Modification of viral coat proteins to enable cell specific delivery of gene of interest is a promising new technique. By altering the surface proteins of candidate viral systems, such as the knob protein of the adenovirus, delivery of genes to specific cells populations of the inner ear may be possible with limited introduction to adjacent cells. However, little information is known of cell specific epitopes within the inner ear that would allow this type of cell specific targeting of gene therapy.

In addition to gene therapy, the delivery of therapeutic small molecules and/or macromolecules into the inner ear currently relies on non-targeted deliver. For example, therapeutic molecules can be introduced into the inner ear; however, the molecules are not targeted to the cells of the inner ear, such as the spiroganglia, hair cells, lateral wall cells, supporting cells, or the like. As such, the delivery of therapeutic molecules into the inner ear could be benefited from being associated with a targeting moiety, such as an epitope that binds with a receptor on a cell of the inner ear.

Currently, there are no suitable technologies in use in the treatment of hearing and balance disorders. There are no FDA approved drugs for inner ear disease (e.g., sensorineural hearing loss; balance disorders). Development of drug targeting is a key step in developing drugs for the inner ear.

### SUMMARY

Generally, the present invention provides targeting moieties that target the cells of the inner ear. For example, the targeting moieties are polypeptides that have sequences that preferentially target and bind with surface receptors of cells of the inner ear. As such, nucleic acids and/or drugs can be associated with the targeting moieties of the present invention in order to provide therapeutics that are delivered to specific cells in the inner ear. Conjugation of drugs or gene therapy vectors to cell specific peptides may allow the treatment of individual cell types within the inner ear.

In one embodiment, the present invention includes a polypeptide having a sequence of Table 1 in an unnatural configuration. The polynucleotide can either consist of the sequence or include additional polypeptides attached to the ends of the sequences shown in Table 1. The polynucleotide can be in a non-native configuration. For example, the polypeptide is selected from the following: a-h-p-h-h-s-m; h-p-h-h-r-i-f; t-v-p-q-l-t-t; s-t-t-k-l-a-l; m-e-g-y-i-h-r; h-a-i-y-p-r-h; h-s-r-l-l-d-q; i-q-s-p-h-f-f; or y-a-a-h-r-s-h.

In one embodiment, the present invention includes a composition having the polynucleotides that target and interact with receptors on inner ear cell surfaces. Such as composition can include a non-natural component combined with the polypeptide, such combination being unnatural. The non-natural component can be any component or in an amount that is not found in nature with these polypeptides are recited.

In one embodiment, the present invention includes a therapeutic for treating and/or preventing a disease associated with an inner ear of a subject. Such a therapeutic can include: a targeting moiety that targets and interacts with a surface polypeptide of an inner ear cell of the inner ear of the subject; and an active agent linked to the targeting moiety. The linking can be direct or indirect through a linker. The targeting moiety can be an inner ear targeting polypeptide. The active agent can be selected from the group consisting of small molecules, macromolecules, polypeptides, proteins, drugs, nucleic acids, plasmid DNA, siRNA, mRNA, antisense RNA, and combinations thereof.

In one embodiment, the therapeutic is a viral vector. As such, the targeting moiety can be associated with a viral particle. The viral particle can include a nucleic acid as the active agent.

In one embodiment, the present invention includes a method of manufacturing a therapeutic for treating and/or preventing a disease associated with an inner ear of a subject. Such a method can include: providing a targeting moiety that targets and interacts with a surface polypeptide of an inner ear cell of the inner ear of the subject; and associating the targeting moiety with an active agent.

In one embodiment, a manufacturing method can include preparing a polypeptide that targets the inner ear cells of the inner ear as the targeting moiety.

In one embodiment, a manufacturing method can include: preparing a nucleic acid encoding for the polypeptide; introducing the polypeptide into a cell; culturing the cell so as to produce the polypeptide; and purifying the polypeptide from the cell culture.

In one embodiment, the present invention can include a therapeutic composition for treating and/or preventing a disease associated with an inner ear of a subject. Such a therapeutic composition can include: a pharmaceutically acceptable carrier; and a therapeutic associated with the pharmaceutically acceptable carrier, said therapeutic comprising: a targeting moiety that targets and interacts with a surface polypeptide of an inner ear cell of the inner ear of the subject; and an active agent linked to the targeting moiety. The active agent and targeting moieties can be configured as described herein. The pharmaceutically acceptable carrier can be any known pharmaceutical carrier.

In one embodiment, the present invention can include a chromatography column for isolating polypeptides that target inner ear cells. Such a column can include: a support; and a targeting moiety that targets and interacts with a surface polypeptide of an inner ear cell coupled to the support. The targeting moiety can be an inner ear targeting polypeptide as described herein. In one option, the targeting moiety is directly coupled to the support. In another option, the targeting moiety is coupled to the support via a linker.

### DESCRIPTION OF FIGURES

Generally, the present invention provides targeting moieties that target the cells of the inner ear. For example, the targeting moieties are polypeptides that have sequences that preferentially target and bind with surface receptors of cells of the inner ear. As such, nucleic acids and/or drugs can be associated with the targeting moieties of the present invention in order to provide therapeutics that are delivered to specific cells in the inner ear. Conjugation of drugs or gene therapy vectors to cell specific peptides may allow the treatment of individual cell types within the inner ear.

FIG. 1 shows the effects of amplification on phage plaque forming units (pfu) per μl after phage panning of mouse saccule and utricle cultures. Amplification was performed for 4.5 hrs in e. coli after panning saccule and utricle cultures with the pool of phage from the previous round amplification.

FIG. 2 is an image showing immunohistochemistry staining of mouse inner ear with an individual phage colony after four rounds of amplification. Intense staining of the basement membrane is observed in the saccule and utricle with no non-specific binding to surrounding structures.

FIG. 3 is an image showing immunohistochemistry staining of mouse inner ear with an individual phage colony after four rounds of amplification. Intense staining of supporting cells surrounding hair cells is observed.

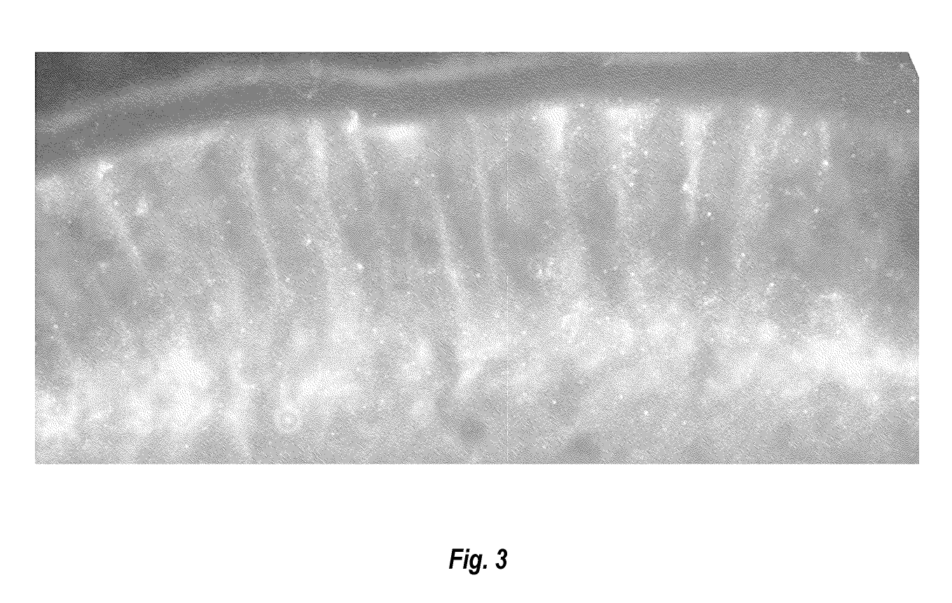

FIGS. 4A-4D show the effect of atoh1 delivery to mice treated with intracochlear nyomycin.

FIG. 5 shows the counts of serially sectioned saccules showed average hair cell counts of 21.8+2/100 μm for untreated controls, 2.2+2/100 μm for aminoglycoside only treated animals and 14.1+2.2/100 μm for aminoglycoside and Ad math1.11D.

FIG. 6 shows that age matched control mice had a baseline swim test time of 10+1.4 seconds.

Table 1 shows phage pIII coat protein —NH₂ terminal 7 amino acid DNA sequences and translation after four rounds of panning of mouse saccule and utricle cultures. After translation of DNA, peptide sequences were queried against the BLAST protein database for homology to known murine proteins.

### DETAILED DESCRIPTION

Generally, the present invention provides targeting moieties that preferentially target the cells of the inner ear. For example, the targeting moieties are polypeptides that have sequences that preferentially target and bind with surface receptors of cells of the inner ear. As such, nucleic acids, polypeptides (e.g., proteins), and/or drugs can be associated with the targeting moieties of the present invention in order to provide therapeutics that are delivered to specific cells in the inner ear. Conjugation of drugs, proteins, or gene therapy vectors to cell specific peptides may allow the treatment of individual cell types within the inner ear. Also, the targeting peptides can be coupled to liposomes, gene carriers, as well as small molecules or proteins for providing the coupled cargo to the inner ear cells.

Currently, there is no successful way to specifically target the complex cells of the inner ear in order to provide a therapeutic cargo to these cells. The present invention uses various polypeptide sequences in order to target and bind with cell surface receptors on specific cell types in the inner ear. This will allow the targeted and preferential delivery of therapeutic molecules (e.g., molecules, drug-carrier complexes, viral vectors, polypeptides, etc.) to be preferentially delivered to specific cells in the inner ear. Potential applications include the treatment of a variety of acquired and congenital inner ear diseases, and many other uses.

Accordingly, the targeting moieties of the present invention include a series of polypeptides that are designed to target specific cells in the inner ear. The specific polypeptide sequences are shown in Table 1 and the Sequence Listing. These polypeptide sequences are a series of peptides that bind bioavailable epitopes in the inner ear, and were designed using phage display.

Phage display is a well known technique in which a library of variants of a peptide is expressed on the outside of a phage virion, while the genetic material encoding each variant resides on the inside. This creates a physical linkage between each variant protein sequence and the DNA encoding it, which allows rapid partitioning based on binding affinity to a given target molecule by an in vitro selection process called “panning.” As such, one of ordinary skill in the art could utilize the present disclosure in order to identify other targeting moieties specific for the inner ear cells.

The targeting moieties were designed and prepared as described herein. Briefly, three libraries of M13 bacteria phage consisting of 7 random amino acids, 12 amino acids or 7 amino acids flanked by a pair of cysteine residues to create a binding loop attached to the N-terminus of the minor coat protein pIII were prepared. These libraries were screened against saccule and utricle cultures isolated from C57B1/6 mice. After binding to the tissue in vitro, the phage were eluted and amplified in E. coli. Additional binding/amplification cycles were performed to enrich the pool in favor of binding sequences. After 3 rounds of amplification, individual clones were characterized by DNA sequencing. The sequences were queried against protein databases for homology to sequencing. The sequences were queried against protein databases for homology to known inner ear proteins. Immunofluorescent staining of individual phage clones was performed after binding to macular cultures to localize each clones binding site.

The data demonstrate that phage panning of mouse saccule and utricle isolates generates ear-cell specific amino acid sequences and selective binding to specific cell populations within the mouse inner ear. These characterized sequences can allow for selective binding and delivery of therapeutics to cells within the inner ear.

While any of the polypeptides of Table 1 may be useful, specific polypeptides have been identified that may improve the targeting efficacy. These specific polypeptides can include: a-h-p-h-h-s-m; h-p-h-h-r-i-f; t-v-p-q-l-t-t; s-t-t-k-l-a-l; e-g-y-i-h-r; h-a-i-y-p-r-h; h-s-r-l-l-d-q; i-q-s-p-h-f-f; and/or y-a-a-h-r-s-h. Any of these specific polypeptides can be used in the therapeutic, diagnostic, purification, or chromatography methods described herein. Also, these polypeptides can be incorporated into other polypeptides, such as surface proteins on a viral capsid. This can allow a virus vector to be configured for preferentially targeting the inner ear cells.

Implementation of the invention can also involve synthesis or other preparation of targeting polypeptides. The targeting polypeptides can then be conjugated to other molecules, delivery vehicles, gene carriers, or other therapeutics. Also, the targeting peptides can be configured to be encoded by a viral vector so that the polypeptides are expressed on the viral vector for preferentially targeting the inner ear.

The therapeutics of the present invention include an active agent (e.g., nucleic acid, polypeptide, small molecule, macromolecule, drug, and the like) associated with a targeting moiety that preferentially targets inner ear cells. As such, the therapeutics can be configured for treating any disease associated with the inner ear or infecting the inner ear. For example, the therapeutics can treat, inhibit, and/or prevent hearing loss, balance disorders, infection, and the like.

In one embodiment, the targeting moieties of the present invention can be used for chromatographic separation and purification of cells that have receptors for the targeting ligands or for separation and purification of the proteins that function as receptors for the inner ear targeting polypeptides of the present invention. For example, a chromatography column can include a chromatography composition having at least one of the polypeptide targeting moieties of Table 1. Preparing chromatography columns to include targeting moieties of the present invention is well within the skill of one of ordinary skill in the art. The chromatography composition can be configured into any suitable separation or purification composition.

In one embodiment, the present invention can be used with as a diagnostic reagent. That is, the inner ear cell targeting polypeptides can be associated with a diagnostic agent so that the diagnostic agent can target the inner ear. For example, the diagnostic can be radiopaque substance that can be viewed via fluoroscopy or x-ray. This can help identify the inner ear via imaging. Other diagnostic techniques well known in the art can be applied by associating the polypeptides with the diagnostic substances. The targeting moiety can be used in any diagnostic composition and can preferentially locate a diagnostic agent to inner ear cells.

In one embodiment, the present invention can include the polynucleotides and/or polypeptides of Table 1 or a composition having the same. These polypeptides are not in a naturally occurring state, and are significantly different from any protein from which they are obtained by virtue of haying an N-terminus at one end of the polypeptide and a C-terminus at the other end of the polypeptide. In their natural occurrence, both ends of these polypeptides would be coupled to other amino acids through peptide linkages, and it well understood that N-terminus and C-terminus end configurations are different from peptide linkages. Also, the N-terminus and C-terminus end configurations allow for the polypeptides to have 3-dimensional conformations that are not possible when the ends of these polypeptides are coupled to additional polypeptides through peptide bonds as found in nature. The polynucleotides can be included into expression vectors so that the polypeptides are expressed in either bacteria, viruses, or other cells. The expression vectors are not natural, and thereby an expression vector encoding for the polypeptides is different from the natural gene sequences that encode for the naturally occurring forms of these polypeptides. Moreover, the cDNA of the polynucleotide sequences that encode for the polypeptides of Table 1 are not natural. While the specific polynucleotide coding sequence for each polypeptide of Table 1 may be found in nature, the natural form of the polynucleotides are inherently coupled to additional polynucleotide stretches at both the 5′ and 3′ ends. Such natural coupling of the 5′ and 3′ ends of the polynucleotides to other natural polynucleotide stretches results in the polynucleotides of Table 1 being different from the natural configuration because free 5′ and 3′ ends of the polynucleotides are chemically different from the bonds formed with adjacent nucleotides. As such, the chemical structure of the polynucleotides of Table 1, particularly at the 5′ and 3′ ends, is different from the ends of these polynucleotides as found in nature. Furthermore, the 3-dimensional conformation of the polynucleotides in Table 1 is significantly different from the 3-dimensional conformation found in nature.

In one embodiment, the present invention includes one or more polypeptides of sequence: a-h-p-h-h-s-m; h-p-h-h-r-i-f; t-v-p-q-l-t-t; s-t-t-k-l-a-l; e-g-y-i-h-r; m-e-g-y-i-h-r; h-a-i-y-p-r-h; h-s-r-l-l-d-q; i-q-s-p-h-f-f; and/or y-a-a-h-r-s-h. These polypeptides can be included in various compositions with other components, such as components not found in nature associated with these polypeptides. On aspect includes a non-naturally occurring composition having these polypeptides. Another aspect includes these polypeptides being in a non-native state, such as being separated from the natural protein from which they occur by cleaving the peptide bonds and forming an N-terminus and/or C-terminus on the polypeptide ends.

In one embodiment, the present invention includes one or more polynucleotides that encode for one or more polypeptides of sequence: a-h-p-h-h-s-m; h-p-h-h-r-i-f; t-v-p-q-l-t-t; s-t-t-k-l-a-l; e-g-y-i-h-r; m-e-g-y-i-h-r; h-a-i-y-p-r-h; h-s-r-l-l-d-q; i-q-s-p-h-f-f; and/or y-a-a-h-r-s-h. These polynucleotides can be included in various compositions with other components, such as components not found in nature associated with these polynucleotides. On aspect includes a non-naturally occurring composition having these polynucleotides. Another aspect includes these polynucleotides being in a non-native state, such as being separated from the natural gene from which they occur by cleaving the bonds. In another aspect, the polynucleotides can be included in an expression vector that is not natural. The expression vector can include a promoter sequence or other sequences not found in the natural gene having the polynucleotide. In yet another aspect, the polynuceotides can be included in a non-natural plasmid. In still yet another, the polynucleotides can be included in a bacteria or virus and configured for expressing the polypeptides that are encoded.

In one embodiment, the present invention can include a therapeutic composition for treating, inhibiting, and/or preventing a disease associated with an inner ear of a subject. The composition can include a pharmaceutically acceptable carrier and a therapeutic having an inner ear targeting moiety. The therapeutic can be associated with the pharmaceutically acceptable carrier, and can include the inner ear targeting moiety associated with an active agent. The targeting moiety can be configured to interact with a surface polypeptide of an inner ear cell of the inner ear of the subject. The active agent can be associated with the targeting moiety by any manner. For example, the targeting moiety can be linked to the active agent or a carrier having the active agent. Such linking can be performed by standard conjugation chemistry which is reactant dependent, and such linking can be designed and performed after analysis of the reactants, such as the targeting moiety, therapeutic agent, carrier, linker, and/or other feature.

In one embodiment, the present invention includes a therapeutic for treating, inhibiting, and/or preventing a disease associated with an inner ear of a subject, where the therapeutic has a targeting moiety linked to an active agent. The targeting moiety can be selected to preferentially interact with a surface polypeptide or receptor of an inner ear cell of the inner ear of the subject. The targeting moiety can bind with the inner ear cell so as to induce endocytosis so that the therapeutic is taken into the cell within an endosome.

In one embodiment, the targeting moiety is a polypeptide, such as those recited in Table 1. Other targeting moieties are possible, and such targeting moiety may include about 7 amino acids for interacting with a receptor on a cell of the inner ear. For example, the targeting moiety can be a polypeptide selected from: a-h-p-h-h-s-m; h-p-h-h-r-i-f; t-v-p-q-l-t-t; s-t-t-k-l-a-l; e-g-y-i-h-r; m-e-g-y-i-h-r; h-a-i-y-p-r-h; h-s-r-l-l-d-q; i-q-s-p-h-f-f; and/or y-a-a-h-r-s-h. The targeting moiety can include any intact amino acid sequence found in Table 1 or the Sequence Listing alone or in association with other polypeptides.

In one embodiment, the active agent can be selected from the group consisting of small molecules, macromolecules, polypeptides, proteins, drugs, nucleic acids, plasmid DNA, siRNA, mRNA, antisense RNA, viral vectors, viral particles, liposomes carrying the active agent, and combinations thereof as well as any other type of active agent that can provide a therapeutic benefit. Optionally, the targeting moiety is directly coupled to the active agent. The conjugation chemistry can be dependent on the amino acid arrangement of the targeting moiety as well as on the active agent to be linked with the targeting moiety. In another option, the targeting moiety is coupled to the active agent via a linker. The linker can be any polypeptide, polynucleotide, C1-C20 alkyl, polymer, or the like as well as any standard of stable or cleavable linker. The linker may also be biodegradable or bio-cleavable, such as linkers configured to degrade in an endosome so as to release the active agent before entering the lysosome. Cleavable linkers are well known as is the conjugation chemistry for using such cleavable linkers in biotherapeutic applications.

In one embodiment, the therapeutic composition can include a pH sensitive motif and/or an endosomal disrupting motif. The pH sensitive motif can be configured to destabilize the endosome and allow for the active agent to escape into the cytosol and avoid degradation in the lysosome. Polyethylenimine and polyhistidine are examples of pH sensitive motifs. An endosomal disrupting motif can be a polypeptide that is configured to disrupt the endosome, and various viral proteins have been identified for such endosomal disruption. An example of an endosomal disrupting motif can include influenza-derived fusogenic peptide diINF-7 or others. The pH sensitive motif and/or an endosomal disrupting motif may be present in the composition and/or may be linked to the targeting moiety and/or therapeutic agent as well as a carrier that carries the therapeutic agent.

In one embodiment, the therapeutic can include a viral vector that has the targeting moiety. Any type of viral vector can be used. When the targeting moiety is a polypeptide such as those listed in Table 1, the targeting moiety can be chemically linked to the viral vector or the DNA encoding for the production of the viral vector can encode for the polypeptide to be present on the surface of the viral vector in a manner sufficient for binding with a receptor on a cell of the inner ear. The targeting moiety can be available as a ligand for an inner ear cell receptor.

In one embodiment, the present invention includes a method of manufacturing a therapeutic that can be used for treating and/or preventing a disease associated with an inner ear of a subject. The manufacturing method can include: providing a targeting moiety that interacts with a surface polypeptide of an inner ear cell of the inner ear of the subject; and associating the targeting moiety with an active agent. This can include chemical conjugation techniques as well as gene expression techniques that result in a therapeutic having the targeting moiety linked to an active agent. These methods can be used to prepare any of the types of therapeutics described herein, and even includes a viral vector being prepared in a cell to have the targeting moiety.

The manufacturing method can include a process for preparing the targeting moiety. When the targeting moiety is a polypeptide, it can be prepared by peptide synthesis techniques or can be prepared by a microbe such as bacteria. The polypeptide can be configured or selected to preferentially target the inner ear cells of the inner ear.

A method for preparing the targeting moiety can include: preparing a nucleic acid encoding for the polypeptide; introducing the polypeptide into a cell; culturing the cell so as to produce the polypeptide; and purifying the polypeptide from the cell culture.

The manufacturing method can also include selecting a particular type of active agent to be associated with the targeting moiety. The active agent can be selected based on the type of malady to be treated, inhibited, or prevented. Some examples of active agents can include small molecules, macromolecules, drugs, polypeptides, proteins, nucleic acids, plasmid DNA, siRNA, mRNA, antisense RNA, viral vectors, carriers containing the active agent, and combinations thereof.

The manufacturing can include coupling the targeting moiety directly to the active agent. As such, standard conjugation chemistry techniques can be used to couple the targeting moiety to the active agent. Otherwise, the targeting moiety can be linked to the active agent via a linker as described herein.

In one embodiment, the present invention can include a chromatography composition having the targeting moiety as described herein. The chromatography composition can be configured to present the targeting moiety so that another composition that comes into contact with the targeting moiety can have polypeptides, proteins, or cells presenting a receptor or receptor portion that interacts with the targeting moiety separated from that composition. The chromatography composition can be used to selectively pull polypeptides, proteins, or cells that bind with the targeting moiety from another composition.

In one example, the chromatography composition can be included in a chromatography column. The chromatography column can be used for isolating receptors or portions of receptors, as well as the cells having the same, that bind with the targeting moiety. As such, the targeting moiety can be bound so some substrate, such as a column, and a composition can be passed over the targeting moiety for selectively binding with polypeptides that bind with the targeting moiety. The polypeptides as well as cells having the same can be separated from the targeting moiety and purified.

In one embodiment, the peptides of the present invention can be used as research reagents to identify cell types within the inner ear for research or diagnostic purposes. The peptides can specifically target select cell types as described herein. As such, the peptides can be used to identify the presence of the cell types, as well as location. The peptides can be included fusion peptides with a reporter motif or can be conjugated to a reporter moiety for research and/or diagnostic applications.

In one embodiment, the peptides of the present invention can be used to purify specific populations of cells within the inner ear. The purification can allow the specific cells to be available for tissue culture and/or research applications. The peptides can be conjugated to column structures, microspheres, or the like in order to be available for binding to the cells, and then extraction of the bound cells from a composition.

In one embodiment, a polypeptide that binds with the targeting moiety can then be bound to a support, which in turn can be used for isolating additional targeting moieties. Accordingly, additional targeting moieties can then be identified by use of receptors for the targeting moieties described herein in association with a chromatography column or other separation or purification system.

In one embodiment, a therapeutic having a targeting moiety and an active agent can be used in a therapeutic protocol for a malady associated with the inner ear. Accordingly, the therapeutic can be administered in an effective amount for treating, inhibiting, and/or preventing the malady or symptoms associated therewith.

As used herein, the terms “an effective amount”, “therapeutic effective amount”, or “therapeutically effective amount” shall mean an amount or concentration of a compound according to the present invention which is effective within the context of its administration or use. Thus, the term “effective amount” is used throughout the specification to describe concentrations or amounts of compounds according to the present invention which may be used to produce a favorable change in the disease or condition treated, whether that change is a remission, a decrease in the disease manifestation, increase in viability, a favorable physiological result, a reduction in the progress of the disease, or the like, depending upon the disease or condition treated.

As used herein, the term “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the specification and claims includes both one and more than one such excipient. The therapeutic described herein can be formulated with a pharmaceutically acceptable excipient.

As used herein, the term “co-administration” or “combination therapy” is used to describe a therapy in which at least two active compounds in effective amounts are used to treat breast and/or ovarian tumors. Although the term coadministration preferably includes the administration of two active compounds to the patient at the same time, it is not necessary that the compounds be administered to the patient at the same time, although effective amounts of the individual compounds will be present in the patient at the same time. Accordingly, the therapeutics described herein can be co-administered with other active agents, or multiple therapeutic agents can be co-administered.

Compounds according to the present invention may be used in pharmaceutical compositions having biological/pharmacological activity for the treatment of diseases of the inner ear. These compositions comprise an effective amount of any one or more of the active agents associated with the targeting moiety, optionally in combination with a pharmaceutically acceptable additive, carrier, or excipient. Such a composition can be useful to prevent, alleviate, eliminate, or delay the onset diseases associated with the inner ear, and thereby can be used as a prophylactic or treatment for inner ear diseases.

As used herein, the term “treating” or “treatment” of a disease includes: (a) preventing the disease, i.e. causing the clinical symptoms of the disease not to develop in a mammal that may be exposed to or predisposed to the disease but does not yet experience or display symptoms of the disease; (b) inhibiting the disease, i.e., arresting or reducing the development of the disease or its clinical symptoms; or (c) relieving the disease, i.e., causing regression of the disease or its clinical symptoms.

As used herein, a “subject” or a “patient” refers to any mammal (preferably, a human), and preferably a mammal that may be susceptible to a disease associated with inner ear. This can include diseases with aberrant expression of a gene or genes that can be corrected with gene therapy. Examples include a human, a non-human primate, a cow, a horse, a pig, a sheep, a goat, a dog, a cat or a rodent such as a mouse, a rat, a hamster, or a guinea pig. Generally, the invention is directed toward use with humans.

The amount of the active agent or therapeutic in a formulation can vary within the full range employed by those skilled in the art. Typically, the formulation will contain, on a weight percent basis, from about 0.01-99.99 weight percent of the compounds of the present invention based on the total formulation, with the balance being one or more suitable pharmaceutical excipients. Preferably, the therapeutics are present at a level of about 1-80 weight percent.

Pharmaceutical preparations include sterile aqueous or non-aqueous solutions, suspensions and emulsions. Examples of non-aqueous solvents are propylene glycol, polyethylene glycol, vegetable oil such as olive oil, an injectable organic esters such as ethyloliate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers, (such as those based on Ringer's dextrose), and the like. Preservatives and other additives may also be present such as, for example, antimicrobials, antioxidants, chelating agents and inert gases and the like. Those of skill in the art can readily determine the various parameters for preparing these pharmaceutical compositions without resort to undue experimentation.

Pharmacological compositions may be prepared from water-insoluble compounds, or salts thereof, such as aqueous base emulsions. In such embodiments, the pharmacological composition will typically contain a sufficient amount of pharmaceutically acceptable emulsifying agent to emulsify the desired amount of the pharmacological agent. Useful emulsifying agents include, but are not limited to, phosphatidyl cholines, lecithin, and the like.

The pharmaceutically acceptable excipients, such as vehicles, adjuvants, carriers or diluents, are readily available to the public. Examples of suitable excipients can include, but are not limited to, the following: acidulents, such as lactic acid, hydrochloric acid, and tartaric acid; solubilizing components, such as non-ionic, cationic, and anionic surfactants: absorbents, such as bentonite, cellulose, and kaolin; alkalizing components, such as diethanolamine, potassium citrate, and sodium bicarbonate; anticaking components, such as calcium phosphate tribasic, magnesium trisilicate, and talc; antimicrobial components, such as benzoic acid, sorbic acid, benzyl alcohol, benzethonium chloride, bronopol, alkyl parabens, cetrimide, phenol, phenylmercuric acetate, thimerosol, and phenoxyethanol; antioxidants, such as ascorbic acid, alpha tocopherol, propyl gallate, and sodium metabisulfite; binders, such as acacia, alginic acid, carboxymethyl cellulose, hydroxyethyl cellulose; dextrin, gelatin, guar gum, magnesium aluminum silicate, maltodextrin, povidone, starch, vegetable oil, and zein; buffering components, such as sodium phosphate, malic acid, and potassium citrate; chelating components, such as EDTA, malic acid, and maltol; coating components, such as adjunct sugar, cetyl alcohol, polyvinyl alcohol, carnauba wax, lactose maltitol, titanium dioxide; controlled release vehicles, such as microcrystalline wax, white wax, and yellow wax; desiccants, such as calcium sulfate; detergents, such as sodium lauryl sulfate; diluents, such as calcium phosphate, sorbitol, starch, talc, lactitol, polymethacrylates, sodium chloride, and glyceryl palmitostearate; disintegrants, such as colloidal silicon dioxide, croscarmellose sodium, magnesium aluminum silicate, potassium polacrilin, and sodium starch glycolate; dispersing components, such as poloxamer 386, and polyoxyethylene fatty esters (polysorbates); emollients, such as cetearyl alcohol, lanolin, mineral oil, petrolatum, cholesterol, isopropyl myristate, and lecithin; emulsifying components, such as anionic emulsifying wax, monoethanolamine, and medium chain triglycerides; flavoring components, such as ethyl maltol, ethyl vanillin, fumaric acid, malic acid, maltol, and menthol; humectants, such as glycerin, propylene glycol, sorbitol, and triacetin; lubricants, such as calcium stearate, canola oil, glyceryl palmitostearate, magnesium oxide, poloxymer, sodium benzoate, stearic acid, and zinc stearate; solvents, such as alcohols, benzyl phenylformate, vegetable oils, diethyl phthalate, ethyl oleate, glycerol, glycofurol, for indigo carmine, polyethylene glycol, for sunset yellow, for tartazine, triacetin; stabilizing components, such as cyclodextrins, albumin, xanthan gum; and tonicity components, such as glycerol, dextrose, potassium chloride, and sodium chloride; and mixture thereof. Excipients include those that alter the rate of absorption, bioavailability, or other pharmacokinetic properties of pharmaceuticals, dietary supplements, alternative medicines, or nutraceuticals.

Other examples of suitable excipients, binders and fillers are listed in Remington's Pharmaceutical Sciences, 18th Edition, ed. Alfonso Gennaro, Mack Publishing Co. Easton, Pa., 1995 and Handbook of Pharmaceutical Excipients, 3rd Edition, ed. Arthur H. Kibbe, American Pharmaceutical Association, Washington D.C. 2000, both of which are incorporated herein by reference.

In general, therapeutics of this invention can be administered as pharmaceutical compositions by any one of the following routes: oral, systemic (e.g., transdermal, intranasal or by suppository), directly into the inner ear, or parenteral (e.g., intramuscular, intravenous or subcutaneous) administration. One manner of administration is oral using a convenient daily dosage regimen which can be adjusted according to the degree of affliction. Compositions can take the form of tablets, pills, capsules, semisolids, powders, sustained release formulations, solutions, suspensions, elixirs, aerosols, or any other appropriate compositions. Another manner for administering compounds of this invention is inhalation.

The administration may be localized in the inner ear (i.e., to a particular region, physiological system, tissue, organ, or cell type of the inner ear) or systemic. For example, the composition may be administered through parental injection, implantation, orally, vaginally, rectally, buccally, pulmonary, topically, nasally, transdermally, surgical administration, or any other method of administration where access to the target by the composition is achieved. Examples of parental modalities that can be used with the invention include intravenous, intradermal, subcutaneous, intracavity, intramuscular, intraperitoneal, epidural, or intrathecal. Examples of implantation modalities include any implantable or injectable drug delivery system. Oral administration may be used for some treatments because of the convenience to the patient as well as the dosing schedule. Compositions suitable for oral administration may be presented as discrete units such as capsules, pills, cachettes, tables, or lozenges, each containing a predetermined amount of the active compound. Other oral compositions include suspensions in aqueous or non-aqueous liquids such as a syrup, an elixir, or an emulsion.

The active agents can be encapsulated in a vehicle such as liposomes, such as liposomes having the targeting moiety linked thereto. The liposomes can be preferentially directed to the inner ear cells via the targeting moiety. The liposomes can facilitate transfer of the active agents into the targeted tissue, as described, for example, in U.S. Pat. No. 5,879,713 to Roth et al. and Woodle, et al., U.S. Pat. No. 5,013,556, the contents of which are hereby incorporated by reference. The compounds can be targeted by selecting an encapsulating medium of an appropriate size such that the medium delivers the molecules to a particular target. For example, encapsulating the compounds within microparticles, preferably biocompatible and/or biodegradable microparticles, which are appropriate sized to infiltrate, but remain trapped within, the capillary beds and alveoli of the lungs can be used for targeted delivery to these regions of the body following administration to a patient by infusion or injection.

Microparticles can also be prepared to have the active agent and present the targeting moiety for preferential delivery to inner ear cells. Microparticles can be fabricated from different polymers using a variety of different methods known to those skilled in the art. The solvent evaporation technique is described, for example, in E. Mathiowitz, et al., J. Scanning Microscopy, 4, 329 (1990); L. R. Beck, et al., Fertil. Steril., 31, 545 (1979); and S. Benita, et al., J. Pharm. Sci., 73, 1721 (1984). The hot-melt microencapsulation technique is described by E. Mathiowitz, et al., Reactive Polymers, 6, 275 (1987). The spray drying technique is also well known to those of skill in the art. Spray drying involves dissolving a suitable polymer in an appropriate solvent. A known amount of the compound is suspended (insoluble drugs) or co-dissolved (soluble drugs) in the polymer solution. The solution or the dispersion is then spray-dried. Microparticles ranging between 1-10 microns are obtained with a morphology which depends on the type of polymer used.

The compositions of the present invention may be given in dosages, generally at the maximum amount while avoiding or minimizing any potentially detrimental side effects. The compositions can be administered in effective amounts, alone or in a cocktail with other compounds, for example, other compounds that can be used to treat and/or prevent inner ear diseases. An effective amount is generally an amount sufficient to inhibit inner ear diseases within the subject.

In one embodiment of the present invention, therapeutically effective amounts of compounds of the present invention may range from approximately 0.05 to 50 mg per kilogram body weight of the recipient per day; preferably about 0.01-25 mg/kg/day, more preferably from about 0.5 to 10 mg/kg/day. Thus, for administration to a 70 kg person, the dosage range would most preferably be about 35-70 mg per day.

In one embodiment, a catheter or catheter-like medical device is used to direct the composition directly to the location of the inner ear. As will be readily apparent to one skilled in the art, the useful in vivo dosage to be administered and the particular mode of administration will vary depending upon the age, weight and mammalian species treated, the particular therapeutics employed, and the specific use for which these therapeutics are employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

In one embodiment, the present invention includes the active agent being a polynucleotide. As such, the polynucleotide can be delivered via a polynucleotide carrier that is associated with the targeting moiety. This includes polynucleotide carriers having the targeting moiety that can target the cells of the inner ear and transport the siRNA across a cell membrane. Polynucleotide carriers are well known in the art of cellular nucleic acid delivery. Preferred polynucleotide carriers include polymers, lipids, lipopolymers, lipid-peptide mixtures, and the like that are capable of complexing with a polynucleotide and delivering the polynucleotide into a cell in a manner that retains the gene silencing functionality without being overly toxic.

In one embodiment, the polynucleotide carrier is a viral vector or viral particle. As such, the targeting moiety is associated with the viral particle having the polynucleotide contained therein. Viral particles for use in delivering therapeutic polypeptides are well known in the art.

In one embodiment, the therapeutic includes an Atoh1 gene or other expression vector as the active agent. The Atoh1 gene can be associated with any gene carrier configured for delivery in the inner ear, and has a targeting moiety that preferentially targets the inner ear. Also, the Hath1 gene can be used at the active agent. The therapeutic can deliver Atoh1 and/or hath1 genes to the inner ear cells. These genes can then facilitate regeneration of vestibular hair cells. Vestibular hair cell regeneration using atoh1 and/or Hath1 therapy can be obtained by polymeric (e.g., polyethylenimine) or viral vector (e.g., adeno-associated virus). One example of a viral vector can be an advanced generation Ad 5 vector (e.g., with E1, E3, E4 deleted) that expresses the human homolog of atoh1 using an hCMV promoter.

Other strategies can be used to regenerate hair cells, such as for example one or more of: inhibition of Hes 1; inhibition of Hes 5; inhibition of p27-kip 1; dual expressing vectors carrying atoh1 and expression of dsRNAi genes active in hair cell differentiation and survival such as pou4f3; hath1 mRNA; Atoh1 mRNA; or others.

Although there are a diverse series of underlying etiologies for balance disorders, loss of vestibular hair cells represents a common cause of balance dysfunction. Other than rehabilitation there are no directed therapies aimed at recovery of vestibular function. As such, the therapeutic can be configured for repairing or avoiding balance dysfunction.

In one embodiment, the therapeutic can be configured to treat, inhibit, and/or prevent bilateral vestbibular hypofunction (BVH). Severe BVH results in permanent chronic balance dysfunction and oscillopsia. Recovery from vestibular loss may only be possible through replacement of the missing vestibular sensory cells, the gene therapy can be configured to aid in the generation of new vestibular sensory cells.

In one embodiment, the therapeutic can be configured to treat, inhibit, and/or prevent vestibular hypofunction. Vestibular hypofunction can be due to aminoglycoside toxicity, and a therapy can be designed to treat, inhibit, and/or prevent aminoglycoside toxicity.

### Examples

### Isolation of Mouse Saccule and Utricle Cultures

C57B1/6 Mice were purchased from Jackson Laboratory (Bar Harbour, Me.). At 8 weeks of age the mice were sacrificed and saccule and utricles were isolated under direct visualization. The cells were cultured in 100 μl DMEM/0.001 M neomycin for 48 hours as is standard. All animal protocols were approved by the University of Kansas animal research advisory committee.

### Phage Display

A phage library (New England Biolabs, Beverly, Mass.) displaying random 7-mer peptides attached to the NH₂-terminus of the pIII protein of the filamentous M13 bacteriophage was used for panning with mouse inner ear cells. In round 1 of panning, 2×10¹¹ plaque-forming units of the phage library in 200 μl of 1×PBS buffer were added to the inner ear cells and were incubated for 4 h at 4° C. with shaking. The cells were washed 10× with Tris-buffered saline/0.1% Tween under direct visualization and the remaining bound phage were eluted for 10 min with 100 μl of 0.2 M glycine-HCl, pH 2.2, and neutralized with 15 μl of 1 M Tris-HCl, pH 9. A 10 μl aliquot was used for tittering and the remaining eluate was used to infect early log phase ER2738 host bacteria in 20 ml LB media and amplified for 4.5 h at 37° C. with shaking. Bacteria were removed by pellet centrifugation 2×10 min at 10,000 g. The supernantant was added to ⅙ quantity 20% (w/v) polyethylene glycol-8000, 2.5 M NaCl. After overnight incubation at 4° C., the phage was pelleted by centrifugation at 15 min at 10,000 g, and resuspended in 200 μl TBS. Amplified phage product was tittered on X-gal/IPTG plates. A 10 μl aliquot from the phage pool was used to titer phage pfu/μl before and after phage amplification in E. coli. In subsequent rounds 2×10¹¹ plaque-forming units of the amplified phage pool from the previous round were added to mouse inner ear cells and panned was performed as previously described. All panning experiments were performed in duplicate.

### Characterization of Phage Display Library

After 4 rounds of panning, isolated colonies were streak plated on X-gal/IPTG plates. Individual colonies were used to infect early log phase ER2738 host bacteria in 4 ml LB media and amplified for 4.5 h at 37° C. The bacteria was pelleted by centrifugation and phage recovered from the supernatant using QIAprep Spin M13 kit (Qiagen, Valencia, Calif.) and resuspended in 50 μl molecular grade water. ssDNA quantity was determined by absorption spectrophotometry. The 7-mer peptide DNA coding regions were sequenced using a −96 gIII sequencing primer (5′-CCC TCA TAG TTA GCG TAA CG-3′). These sequences were translated and queried on the BLAST protein database (NCBI).

### Immunohistochemistry Identification of Phage Binding Regions in Mouse Inner Ear

Candidate 7-mer peptide epitopes were selected from the BLAST queried sequences. Colonies were amplified in 20 ml LB media and recovered as previously described. Phage (1×10⁹ pfu) were injected into the round window of mice previously treated with 1 μl 0.01 M neomycin injected into the posterior channel 48 hours prior to phage innoculation. Mice were sacrificed by intracardiac perfusion with 4% PBS buffered paraformaldehyde 1 hour after phage inoculation. The temporal bones were isolated and decalcified in Calex (Fisher Scientific, Pittsburgh, Pa.) for 24 hours. The temporal bones were embedded in paraffin and sectioned. Primary antibody directed against the M13 protein (rabbit anti-M13) in 1/100 concentration was added to tissue samples and allowed to incubate for 12 hrs at 4° C. The samples were washed in PBS and incubated with 1/50 concentration fluorescent secondary antibody (goat anti-rabbit, FITC) for 12 hrs at 4° C. After washing, tissue sections were visualized and photographed via fluorescent microscopy.

Amplification of M13 Bacteriophage after Phage Panning

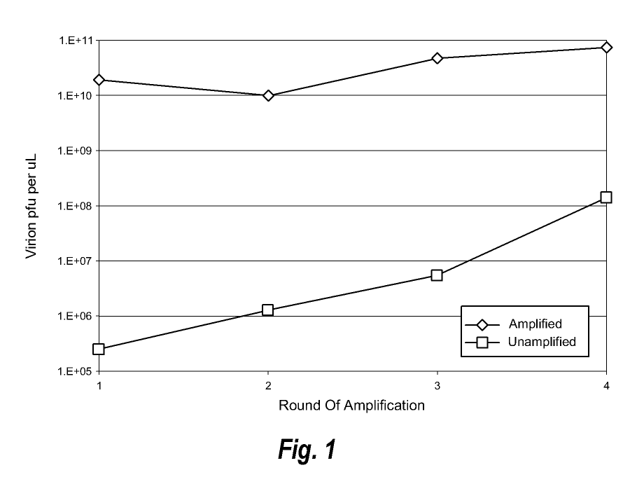

A total of four rounds of phage panning of mouse macular organ cultures were completed. The result from round 1 of panning had 2.53×10⁵ pfu/μl prior to amplification and 1.92×10¹⁰ pfu/μl after amplification to yield a 7.60×10⁴ increase in phage quantity with amplification (FIG. 1). Round 2 of panning had 1.29×10⁶ pfu/μl prior to amplification and 1.01×10¹⁰ pfu/μl after amplification to yield a 7.79×10³ to increase in phage quantity with amplification. Round 3 of panning had 5.50×10⁶ pfu/μl prior to amplification and 4.77×10¹⁰ pfu/μl after amplification to yield a 8.68×10³ increase in phage quantity with amplification. Round 4 of panning had 1.42×10⁸ pfu/μl prior to amplification and 7.47×10¹⁰ pfu/μl after amplification to yield a 5.26×10² increase in phage quantity with amplification.

FIG. 1 shows the effects of amplification on phage plaque forming units (pfu) per μl after phage panning of mouse saccule and utricle cultures. Amplification was performed for 4.5 hrs in e. coli after panning saccule and utricle cultures with the pool of phage from the previous round amplification.

### Characterization of Phage Binding Epitopes to Mouse Inner Ear

One hundred colonies were sequenced (Table 1). There were 23 colonies which translated to the peptide sequence t-v-p-q-l-t-t, which was homologous to the protein laminin. Eleven colonies which translated to the peptide sequence s-t-t-k-l-a-l, which was homologous to a sodium/hydrogen exchanger protein. Eight colonies which translated to the peptide sequence m-e-g-y-i-h-r, which was homologous to the protein cathepsin L. Two colonies each that translated to the peptide sequences h-a-i-y-p-r-h, h-s-r-l-l-d-q, i-q-s-p-h-f-f and y-a-a-h-r-s-h, which were homologous to the proteins itchy 3, discs large homolog, protocadherin and myelin transcription factor 1, respectively. The remaining colony sequences occurred only once, however within these sequences, several translated to peptides that had homology to the same protein. For example, a-h-p-h-h-s-m and h-p-h-h-r-i-f both contained the peptide sequence h-p-h-h with homology to mitogen activated protein kinase 8.

### Immunohistochemical Staining of Phage Binding Epitopes

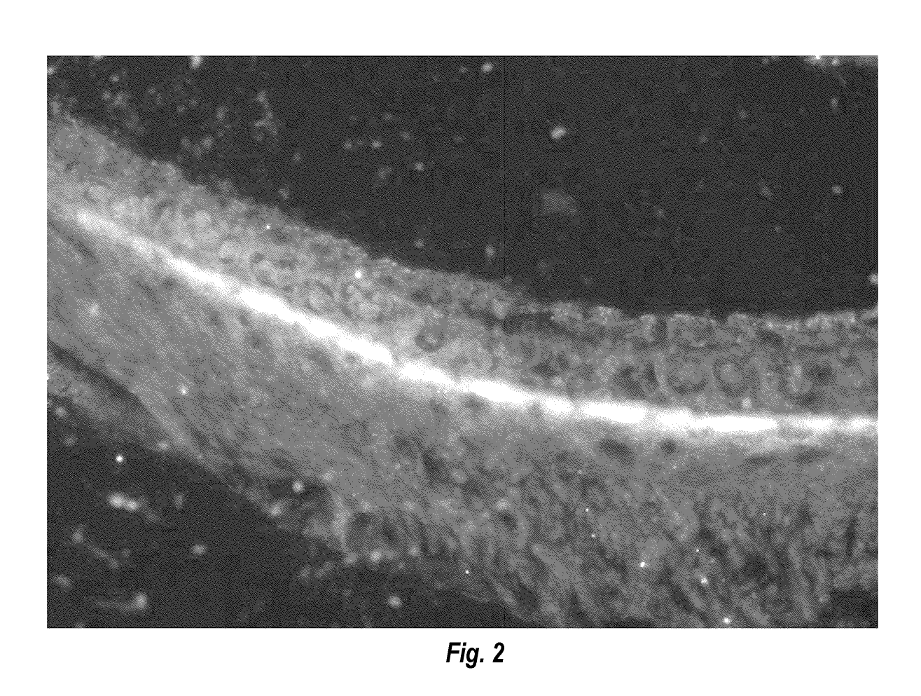

To determine the potential for identified peptide sequences to bind in vitro be purified individual batches of phage and injected them into the perilymph via the round window. The animals were allowed to recover and were then fixed via perfusion. Sections of the inner ear were immunostained for the presence phage to determine if the phage had been able to localize to supporting cells in vivo. As can be seen in FIG. 2 different patterns of phage localization ranging from attachment of the phage to the basement membrane to attachment of the phage to the supporting cells can be seen. In some cases the phage that were developed to bind against vestibular supporting cells also showed cross specific binding to cochlear supporting cells. A summary of common peptide components and binding sites within the inner ear is shown in Table 2. The most common pattern of distribution that was seen was binding to the basement membrane. The majority of the phage particles tested demonstrated either binding to the vestibular supporting cells alone. Additional groups of phage showed more non specific binding to all tissue of the inner ear. Isolated phage also demonstrated absence of binding within the inner ear, suggesting that the ligand or the phage was not bio-available in an in vivo preparation.

TABLE 2

| | Organ of | |
|---|---|---|
| Peptide | Corti | Macular Organs |
| ProThrProMeThrLeuTyr | Pillar | No binding |
| | cells only | |
| HisAlaIleTyrProArgHis | Deiter's | Basement |
| | Cells | membrane only |
| SerGluGlyArgLeuTyrLys or | No binding | Base of sup- |
| AlaLeuGlnTyrHisAsnLeu | | porting cell |
| ThrHisGlnAlaArgSerVal or | No binding | Supporting |
| SerAsnGlnLeuLeuMetThr | | cells |
| ProProArgProIleProMet | No binding | Hair cells and |
| | | supporting |
| | | cells |
| HisProHisHisArgIlePhe | No binding | Supporting |
| | | cells |
| MetProIleProArgProPro | Supporting | Supporting |
| | cells | cells |
| SerThrThrLysLeuAlaLeu | Supporting | Supporting |
| | cells | cells |
| SerThrThrLysLeuAlaLeu | No binding | Supporting |
| | | cells |

### Viral Vectors

The Ad viral system can be used for targeting inner ear cells when having a targeting moiety. The primer and probe sequences are as follows: Forward primer: A5s 3825 5′-CGCGGGATTGTGACTGACT-3′, Reverse primer: A5a 3902 5′-GCCAAAAGAGCCGTCAACTT-3′, Fluorogenic Probe OLIGO 1 5′-FAM-AGCAGTGCAGCTTCCCGTTCATCC-TAMRA-3′. A standard curve is generated using eleven serial dilutions of the pAdE1(L)E3(10)E4(WT) plasmid DNA. Data are collected by the ABI Prism 7700 Sequence Detection System (PE Applied Biosystems). The samples are quantified according to their standard deviation and mean in relation to the standard curve.

The adenovirus vector was prepared to have an adenovector (i.e., adenovirus vector) backbone, which was used for all experiments, where adenovirus regions E1A, E1B, E3, and E4 were deleted. The production system for these adenovectors provides robust replication of the adenovector and purified stocks at 5×10¹¹ to 2×10¹² total particles (particle unit, pu) per ml with a total particle to active particle (fluorescent focus unit, ffu) ratio ranging from 3-10 pu/ffu. Total particles (pu) are determined by a spectrophotmetric assay that has been standardized and qualified to reliably and robustly quantify the total particles within a lot of adenovector. Adenovector lots are purified aliquoted and stored at −80° C. Individual aliquots are used for each experiment to prevent loss of activity associated with freeze thaw cycles. Expression is driven by the hCMV promoter and the expression cassette contains an optimized artificial splice that is 5′ of the open reading frame (ORF) being expressed and an SV40 polyadenylation site and transcriptional stop that is 3′ of the ORF. Production of Fiber/Knob modified vectors is carried out as known in the art.

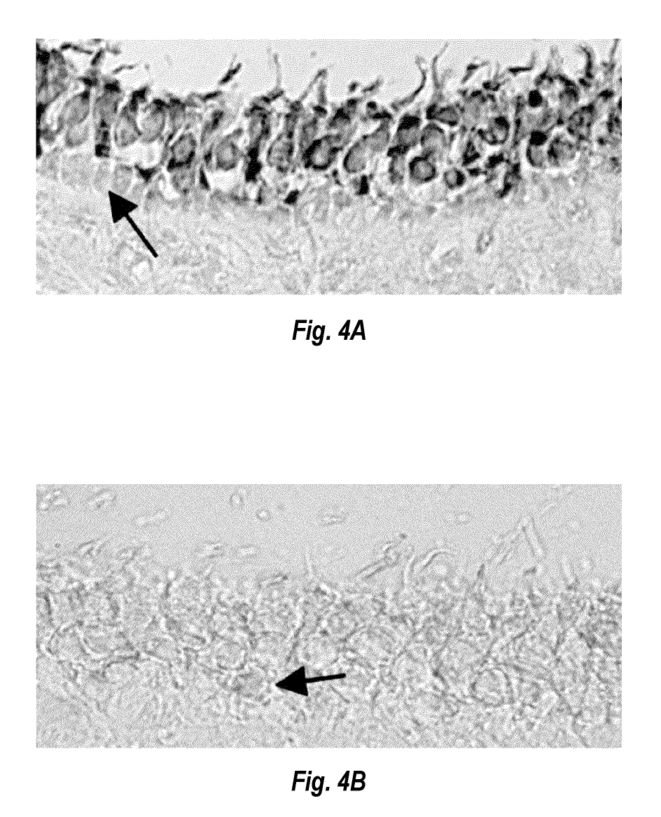

The adenovector can encode for the production of atoh1 gene, which when expressed can induce formation of new hair cells in the vestibular system. This therapy can improve the balance in animals treated with atoh1. It has also been found that adenovector delivery of atoh1 genes to the damaged vestibular system results in regeneration of vestibular hair cells in vivo. Briefly, mice were treated with an injection of neomycin 10⁻³ M into the scala tympani. After 5 days animals were re-anesthetized and 1 μl of Ad.math1.11d (108 pu) were delivered via a fenestration in the posterior semicircular canal. After 4 weeks, animals were sacrificed and then processed for myosin VII immunohistochemistry. Untreated controls (FIG. 4A) show normal distribution of myosin VII in the utricle. Neomycin only treated controls demonstrate little myosin VII labeling at 5 days post aminoglycoside (FIG. 4B). There is no evidence of spontaneous hair cell recovery seen after 1 month (FIG. 4C). Neomycin and atoh 1 treated animals demonstrated myosin VII positive cells in the (FIG. 4D) that have stereocilia (arrow). Myosin VII positive cells can be seen at the base of the neuroepithelium at the level where supporting cell nuclei are normally located. This suggests that atoh1 is not promoting repair but inducing the transformation of transfected cells into cells with the characteristics of hair cells.

Particularly, FIGS. 4A-4D show the effect of atoh1 delivery on mice treated with intracochlear neomycin. Control macular organs (FIG. 4A) demonstrate myosin VII positive hair cells and clearly demarcated supporting cell nuclei (arrow). Five days after injection of neomycin 10⁻³ M to the scala tympani only occasional myosin VII positive cells (arrow) remain and the neuroepithelium is disrupted (FIG. 4B). At one month post aminoglycoside treatment followed after 48 hours with injection of a control vector into the posterior semicircular canal, no spontaneous recovery of myosin VII positive cells is seen (FIG. 4C). Animals injected with neomycin followed after 48 hours with injection of Ad.atoh1.11D show recovery of myosin VII positive cells that have stereocilia (arrow) (FIG. 4D). This demonstrates that atoh1 delivery into the vestibular portion of the inner ear induces recovery of hair cells after aminoglycoside toxicity.

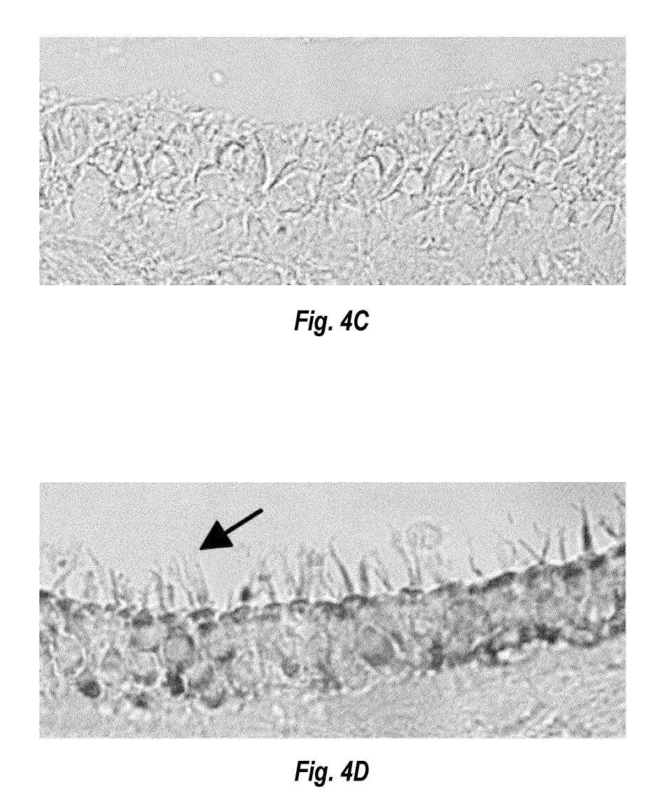

Characterization of atoh1/math1 effects were as follows. Unilateral vestibular damage was induced by injecting 2 μl of neomycin (10⁻³ M) in to the inner ear of mice. After inducing unilateral vestibular dysfunction mice received an intracochlear injection of an advanced generation adenovector carrying atoh1 driven by a human CMV promoter (Admath1.11D) via the round window membrane into the scala tympani. This delivery method was used due to ease of surgical access in the mouse and previous documentation of adenovector delivered transgene in the vestibular neuroeptithelium. After one month recovery, animals were sacrificed and processed for histology. Seven micron serial sections were prepared and all hair cells and supporting cells in a 60× field counted in the macular organs and the horizontal canal crista. Cells were identified by position of the nucleus and every fifth section was counted and expressed as cell number per 100 μm length of neuroepithelium. FIG. 5 shows the counts of serially sectioned saccules showed average hair cell counts of 21.8+2/100 μm for untreated controls (n=5), 2.2+2/100 μm (n=5) for aminoglycoside only treated animals and 14.1+2.2/100 μm (n=20) for aminoglycoside and Ad math1.11D. This represents a statistically significant recovery of hair cells in animals treated with aminoglycoside and Ad math1.11D compared to aminoglycoside only treated animals. Utricles and the horizontal canal crista also showed recovery of vestibular hair cells.

It has been found that Ad.math1.11D induces functional recovery of balance. As a screening test for balance function, mice were swim tested by dropping them from a fixed height into an opaque pool of water. An independent observer, blinded to the treatment the animal had received determined the time it took the animal to initiate purposeful swimming (“swim time”). Animals with impaired vestibular function are known to have to prolonged swim times. Mice were swim tested two months after aminoglycoside ablation followed by no treatment or treatment with Ad math1.11D. Mice treated with atoh1 therapy demonstrated significantly shorter swim times than aminoglycoside only treated animals, which demonstrates a recovery of balance function in the aminoglycoside and atoh1 treated animals. FIG. 5 shows: Vestibular hair cell counts of control, neomycin treated, and neomycin and Admath1.11D treated animals. There is a significant loss of hair cells in the macular organs and the horizontal canal crista of animals treated with intracochlear neomycin (light spotted). Treatment with Admath1.11D after neomycin treatment resulted in a significant restoration of hair cells in the vestibular neuroepithelium compared to neomycin only treated animals.

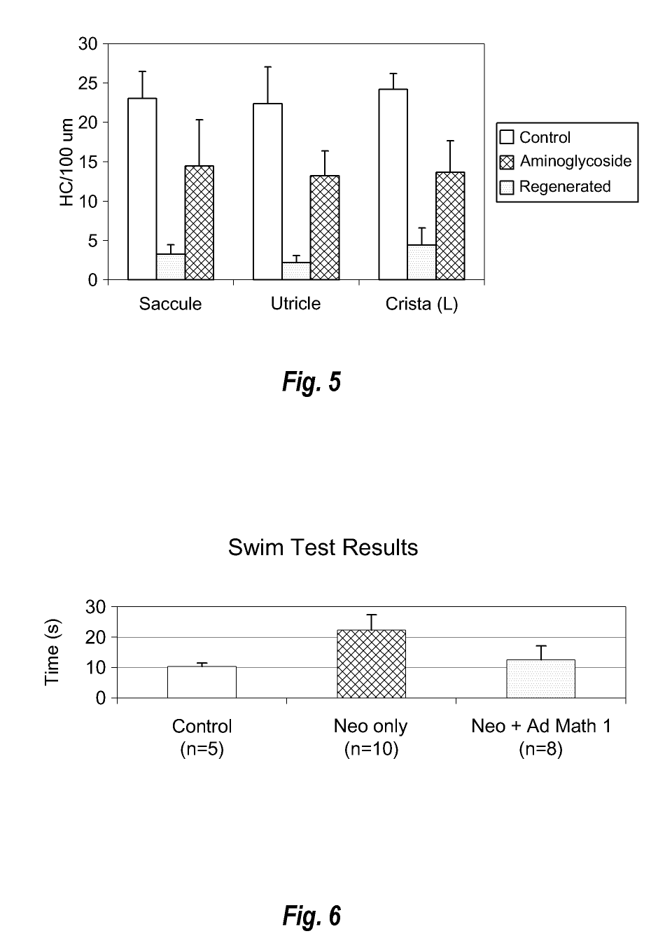

FIG. 6 shows that age matched control mice had a baseline swim test time of 10+1.4 seconds. Aminoglycoside only treated animals showed average swim test times of 22+4.2 seconds. Animals treated with aminoglycoside followed by Admath1.11D showed swim times of 12+3 seconds compared to untreated controls.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope. All references (e.g., publications, journal articles, published patent applications, patents, websites, amino acid sequence identifiers, nucleic acid sequence identifiers, and the like) identified herein are incorporated into this application by specific reference in their entirety.

TABLE 1
Table 1. Phage pIII coat protein —NH₂ terminal 7 amino acid DNA sequences and
translation after four rounds of panning of mouse saccule and utricle cultures. After
translation of DNA, peptide sequences were queried against the BLAST protein database
for homology to known murine proteins.

| # of | | | | | | | | | |
|---|---|---|---|---|---|---|---|---|---|
| Common | DNA Sequencing of 7 amino acid | | | | | | | | BLAST query reults of |
| Sequences | NH2 terminus of pIII Protein | | | | | | | Translation | —NH2 terminus peptide |
| 23 | AGT | AGT | AAG | CTG | AGG | AAC | AGT | tvpqltt | Laminin |
| 11 | CAA | AGC | CAA | CTT | AGT | CGT | CGA | sttklal | Sodium/hydrogen exchanger 9 |
| 8 | CCG | ATG | AAT | ATA | ACC | CTC | CAT | megyihr | Cathepsin L |
| 3 | CGG | AAG | CGG | AGT | CAA | CGG | CAG | rpltplp | dermatan-4-sulfotransferase-1 |
| 2 | ATG | ACG | CGG | ATA | AAT | AGC | ATG | haiyprh | Itchy 3 |
| 2 | CTG | ATC | AAG | CAG | CCT | AGA | ATG | hsrlldq | discs large homolog |
| 2 | AAA | AAA | ATG | AGG | AGA | CTG | AAT | iqsphff | protocadherin |
| 2 | ATG | AGA | ACG | ATG | CGC | AGC | ATA | yaahrsh | myelin transcription factor 1 |
| 1 | CGG | CGT | ACG | CAT | CCT | ATC | CGC | adrmltp | TGF-beta |
| 1 | ATT | ATG | AGA | AAC | CGG | AAA | AGC | afpvshn | connexin |
| 1 | CCA | CGG | CCA | ATC | ATC | ATG | AGC | ahddwpw | follistatin-like 5 |
| 1 | CAT | ACT | ATG | ATG | CGG | ATG | CGC | ahphhsm | Mitogen-activated protein kinase 8 |
| 1 | CAG | ATT | ATG | ATA | CTG | AAG | AGC | alqyhnl | melanocortin-4 receptor |
| 1 | CGA | CCG | ATG | AGG | AGA | AGA | CGC | assphrs | zinc finger |
| 1 | CCG | ATC | CGA | CGG | ATT | CCA | AGC | awnpsdr | Abl1 protein |
| 1 | ACG | ATA | CAC | CGG | AGT | ATT | ATC | dntpvyr | Glutamine fructose-6-phosphate |
| | | | | | | | | | transaminase 1 |
| 1 | CAT | CTT | CAG | CTG | CAG | CGG | CTC | eplnlkm | Vomeromodulin |
| 1 | ATG | CTG | CTG | AGA | CGA | AGG | AAA | fpssqqh | Myosin-9B |
| 1 | CAG | ATG | CGC | ATG | AGC | CGA | AAA | fsahahl | zinc finger protein 68 |
| 1 | AAG | CGG | CGC | ACG | AGT | CTC | CCC | getrapl | calcium channel, voltage-dependent, |
| | | | | | | | | | N type, alpha 1B |
| 1 | ATT | CGT | CCT | CAA | AAC | ATT | CCC | gnvlrtn | Snapc4 protein |
| 1 | ACG | CAG | CGA | CGG | ATT | AGT | ACC | gtnpslr | Aminoacylase 1 |
| 1 | AAC | AAA | AGT | AGG | ATA | CGT | CCC | gtyptfv | adenylosuccinate synthetase 1 |
| 1 | ATA | CAA | AGT | ATT | AAG | ATA | ACC | gylntly | T cell receptor beta chain |
| 1 | AGG | ATA | CAT | AAA | AGC | CGC | ATG | haafmyp | Myosin XVA |
| 1 | CCA | AGC | AGG | ATA | ATT | AGC | ATG | hanypaw | Protein kinase C binding protein |
| 1 | CTG | ATC | AAG | CAG | CCT | AGA | ATG | hfrlldq | cadherin EGF |
| 1 | CCA | CAC | CTG | CGG | AAA | ATT | ATG | hnfpqvw | Chemokine (C-X-C motif) receptor 3 |
| 1 | AAA | AAT | CCG | ATG | ATG | CGG | ATG | hphhrif | Mitogen-activated protein kinase 8 |
| 1 | AGG | CGT | CGT | ATA | AAC | CCT | ATG | hrvyttp | zinc finger protein 451 |
| 1 | AGA | AGG | CAC | CGA | CGC | AAA | AAT | ifasvps | cGMP phosphodiesterase |
| 1 | ACC | CAG | CCT | CGG | AAC | AAT | AAT | iivprlg | olfactory receptor |
| 1 | AAA | AAA | ATG | AGG | AGA | CTG | AAT | iqsphfi | titin isoform N2-B |
| 1 | CTT | CGT | ATA | CAC | CGC | CGG | CTT | kpavytk | trypsinogen |
| 1 | AAG | CAG | AAA | CTC | CGG | AGG | CTT | kppefll | protocadherin-betaO |
| 1 | CGC | CGA | CGG | AAG | AGT | CTG | CTT | kqtlpsa | protein kinase C |
| 1 | ATC | ATA | AGT | CAT | CGG | CGT | CTT | ktpmtyd | zinc fingers and homeoboxes protein 2 |
| 1 | AGC | AGC | AGG | AGA | CGG | ATT | CAT | mnpspaa | serine/threonine kinase 10 |
| 1 | AGG | CGG | CCT | AGG | AAT | AGG | CAT | mpiprpp | G protein-coupled receptor 128 |
| 1 | ATA | AAT | ATC | AGG | ACT | AGG | CAT | mpspdiy | zinc finger protein 710 |
| 1 | CGA | CGC | AGA | CGT | AAA | CAG | CTG | qlftsas | soluble adenylyl cyclase |
| 1 | AGT | ATC | AGG | AAG | AGG | CAT | CTG | qmplpdt | Ropn1I protein |
| 1 | CCG | ATA | AGG | ATG | AGG | AGG | CTG | qpphpyr | Protocadherin 1 |
| 1 | CCG | AGT | AAG | CTG | AGT | AGG | CTG | qptqltr | zinc finger protein 236 |
| 1 | CGT | AGA | CTG | CGG | AAA | CTG | CTG | qqfpqst | cadherin 9 |
| 1 | ATG | CCC | AAC | AGG | CGG | CGA | CTG | qsppvgh | NEDD-4 |
| 1 | ATT | AAG | AAA | CTT | CGG | AGC | CAG | rapkfln | Myosin 6 |
| 1 | CTT | CTG | ATG | ATA | AAG | AGC | ACT | salfhnk | Cpn2 protein |
| 1 | CCA | AGA | AAT | CGG | CGG | ATC | AGA | sdppisw | ATPase, classII, type9A |
| 1 | CTT | ATA | AAG | ACG | CCC | CTC | AGA | segrlyk | Plekha5 protein |
| 1 | ATG | AAT | AAG | ATC | AAG | CAA | ACT | slldlih | ATP-binding cassette |
| 1 | CGT | CAT | AAG | CAG | CTG | ATT | CGA | snqllmt | Ras GTPase-activating protein SynGAP |
| 1 | CGA | AAA | ACC | AAA | ATT | CTG | CGA | sqnfgfs | nucleoporin |
| 1 | CAC | AAC | CAC | ATT | CGA | CCA | CGA | swsnvvv | Paraoxonase 1 |
| 1 | AAC | CGA | ACG | CGC | CTG | ATG | AGT | thqarsv | Wolf-Hirschhorn syndrome protein |

1. A composition comprising:
a polypeptide having a sequence of Table 1 in an unnatural configuration.

2. A composition as in claim 1, wherein the polypeptide is selected from the following:
(SEQ ID NO: 12)
a-h-p-h-h-s-m;
(SEQ ID NO: 29)
h-p-h-h-r-i-f;
(SEQ ID NO: 1)
t-v-p-q-l-t-t;
(SEQ ID NO: 2)
s-t-t-k-l-a-l;
(SEQ ID NO: 3)
m-e-g-y-i-h-r;
(SEQ ID NO: 5)
h-a-i-y-p-r-h;
(SEQ ID NO: 6)
h-s-r-l-l-d-q;
(SEQ ID NO: 7)
i-q-s-p-h-f-f;
or
(SEQ ID NO: 8)
y-a-a-h-r-s-h.

3. A composition as in claim 1, further comprising a non-natural component combined with the polypeptide, such combination being unnatural.

4. A therapeutic for treating and/or preventing a disease associated with an inner ear of a subject, the therapeutic comprising:
a targeting moiety that targets and interacts with a surface polypeptide of an inner ear cell of the inner ear of the subject; and
an active agent linked to the targeting moiety.

5. A therapeutic as in claim 4, wherein the targeting moiety is a polypeptide.

6. A therapeutic as in claim 5, wherein the polypeptide is selected from the polypeptides of Table 1.

7. A therapeutic as in claim 6, wherein the polypeptide is selected from the following:
(SEQ ID NO: 12)
a-h-p-h-h-s-m;
(SEQ ID NO: 29)
h-p-h-h-r-i-f;
(SEQ ID NO: 1)
t-v-p-q-l-t-t;
(SEQ ID NO: 2)
s-t-t-k-l-a-l;
(SEQ ID NO: 3)
m-e-g-y-i-h-r;
(SEQ ID NO: 5)
h-a-i-y-p-r-h;
(SEQ ID NO: 6)
h-s-r-l-l-d-q;
(SEQ ID NO: 7)
i-q-s-p-h-f-f;
or
(SEQ ID NO: 8)
y-a-a-h-r-s-h.

8. A therapeutic as in claim 4, wherein the active agent is selected from the group consisting of small molecules, macromolecules, polypeptides, proteins, drugs, nucleic acids, plasmid DNA, siRNA, mRNA, antisense RNA, and combinations thereof.

9. A therapeutic as in claim 4, wherein the targeting moiety is directly coupled to the active agent.

10. A therapeutic as in claim 4, wherein the targeting moiety is coupled to the active agent via a linker.

11. A therapeutic as in claim 4, wherein the targeting moiety is associated with a viral particle containing the active agent which is a nucleic acid.

12. A therapeutic as in claim 4, wherein the targeting moiety is linked to a carrier which has the active agent.

13. A method of manufacturing a therapeutic for treating and/or preventing a disease associated with an inner ear of a subject, the method comprising:
providing a targeting moiety that targets and interacts with a surface polypeptide of an inner ear cell of the inner ear of the subject; and
associating the targeting moiety with an active agent.

14. A method as in claim 13, further comprising preparing a polypeptide that targets the inner ear cells of the inner ear as the targeting moiety.

15. A method as in claim 13, further comprising:
preparing a nucleic acid encoding for the polypeptide;
introducing the polypeptide into a cell;
culturing the cell so as to produce the polypeptide; and
purifying the polypeptide from the cell culture.

16. A method as in claim 13, wherein the targeting moiety is a polypeptide is selected from the following:
(SEQ ID NO: 12)
a-h-p-h-h-s-m;
(SEQ ID NO: 29)
h-p-h-h-r-i-f;
(SEQ ID NO: 1)
t-v-p-q-l-t-t;
(SEQ ID NO: 2)
s-t-t-k-l-a-l;
(SEQ ID NO: 3)
m-e-g-y-i-h-r;
(SEQ ID NO: 5)
h-a-i-y-p-r-h;
(SEQ ID NO: 6)
h-s-r-l-l-d-q;
(SEQ ID NO: 7)
i-q-s-p-h-f-f;
or
(SEQ ID NO: 8)
y-a-a-h-r-s-h.

17. A therapeutic composition for treating and/or preventing a disease associated with an inner ear of a subject, the composition comprising:
a pharmaceutically acceptable carrier; and
a therapeutic associated with the pharmaceutically acceptable carrier, said therapeutic comprising:
a targeting moiety that targets and interacts with a surface polypeptide of an inner ear cell of the inner ear of the subject; and
an active agent linked to the targeting moiety.

18. A composition as in claim 17, wherein the targeting moiety is a polypeptide.

19. A composition as in claim 18, wherein the polypeptide is selected from the polypeptides of Table 1.

20. A composition as in claim 18, wherein the polypeptide is selected from the following:
(SEQ ID NO: 12)
a-h-p-h-h-s-m;
(SEQ ID NO: 29)
h-p-h-h-r-i-f;
(SEQ ID NO: 1)
t-v-p-q-l-t-t;
(SEQ ID NO: 2)
s-t-t-k-l-a-l;
(SEQ ID NO: 3)
m-e-g-y-i-h-r;
(SEQ ID NO: 5)
h-a-i-y-p-r-h;
(SEQ ID NO: 6)
h-s-r-l-l-d-q;
(SEQ ID NO: 7)
i-q-s-p-h-f-f;
or
(SEQ ID NO: 8)
y-a-a-h-r-s-h.

21. A composition as in claim 17, wherein the active agent is selected from the group consisting of small molecules, macromolecules, polypeptides, proteins, drugs, nucleic acids, plasmid DNA, siRNA, mRNA, antisense RNA, and combinations thereof.

22. A composition as in claim 17, wherein the targeting moiety is directly coupled to the active agent.

23. A composition as in claim 17, wherein the targeting moiety is coupled to the active agent via a linker.

24. A composition as in claim 17, wherein the targeting moiety is associated with a viral particle containing a nucleic acid as the active agent.

25. A chromatography column for isolating polypeptides that target inner ear cells, the column comprising:
a support; and
a targeting moiety that targets and interacts with a surface polypeptide of an inner ear cell coupled to the support.

26. A column as in claim 25, wherein the targeting moiety is a polypeptide.

27. A column as in claim 26, wherein the polypeptide is selected from the polypeptides of Table 1.

28. A column as in claim 26, wherein the polypeptide is selected from the following:
(SEQ ID NO: 12)
a-h-p-h-h-s-m;
(SEQ ID NO: 29)
h-p-h-h-r-i-f;
(SEQ ID NO: 1)
t-v-p-q-l-t-t;
(SEQ ID NO: 2)
s-t-t-k-l-a-l;
(SEQ ID NO: 3)
m-e-g-y-i-h-r;
(SEQ ID NO: 5)
h-a-i-y-p-r-h;
(SEQ ID NO: 6)
h-s-r-l-l-d-q;
(SEQ ID NO: 7)
i-q-s-p-h-f-f;
or
(SEQ ID NO: 8)
y-a-a-h-r-s-h.

29. A column as in claim 25, wherein the targeting moiety is directly coupled to the support.

30. A column as in claim 25, wherein the targeting moiety is coupled to the support via a linker.


SEQUENCE LISTING (127 sequences)

SEQ ID NO: 1 - protein, 7 aa, Artificial Sequence
  Description of Artificial Sequence Synthetic peptide
TVPQLTT

SEQ ID NO: 2 - protein, 7 aa, Artificial Sequence
  Description of Artificial Sequence Synthetic peptide
STTKLAL

SEQ ID NO: 3 - protein, 7 aa, Artificial Sequence
  Description of Artificial Sequence Synthetic peptide
MEGYIHR

SEQ ID NO: 4 - protein, 7 aa, Artificial Sequence
  Description of Artificial Sequence Synthetic peptide
RPLTPLP

SEQ ID NO: 5 - protein, 7 aa, Artificial Sequence
  Description of Artificial Sequence Synthetic peptide
HAIYPRH

SEQ ID NO: 6 - protein, 7 aa, Artificial Sequence
  Description of Artificial Sequence Synthetic peptide
HSRLLDQ

SEQ ID NO: 7 - protein, 7 aa, Artificial Sequence
  Description of Artificial Sequence Synthetic peptide
IQSPHFF

SEQ ID NO: 8 - protein, 7 aa, Artificial Sequence
  Description of Artificial Sequence Synthetic peptide
YAAHRSH

SEQ ID NO: 9 - protein, 7 aa, Artificial Sequence
  Description of Artificial Sequence Synthetic peptide
ADRMLTP

SEQ ID NO: 10 - protein, 7 aa, Artificial Sequence
  Description of Artificial Sequence Synthetic peptide
AFPVSHN

SEQ ID NO: 11 - protein, 7 aa, Artificial Sequence
  Description of Artificial Sequence Synthetic peptide
AHDDWPW

SEQ ID NO: 12 - protein, 7 aa, Artificial Sequence
  Description of Artificial Sequence Synthetic peptide
AHPHHSM

SEQ ID NO: 13 - protein, 7 aa, Artificial Sequence
  Description of Artificial Sequence Synthetic peptide
ALQYHNL

SEQ ID NO: 14 - protein, 7 aa, Artificial Sequence
  Description of Artificial Sequence Synthetic peptide
ASSPHRS

SEQ ID NO: 15 - protein, 7 aa, Artificial Sequence
  Description of Artificial Sequence Synthetic peptide
AWNPSDR

SEQ ID NO: 16 - protein, 7 aa, Artificial Sequence
  Description of Artificial Sequence Synthetic peptide
DNTPVYR

SEQ ID NO: 17 - protein, 7 aa, Artificial Sequence
  Description of Artificial Sequence Synthetic peptide
EPLNLKM

SEQ ID NO: 18 - protein, 7 aa, Artificial Sequence
  Description of Artificial Sequence Synthetic peptide
FPSSQQH

SEQ ID NO: 19 - protein, 7 aa, Artificial Sequence
  Description of Artificial Sequence Synthetic peptide
FSAHAHL

SEQ ID NO: 20 - protein, 7 aa, Artificial Sequence
  Description of Artificial Sequence Synthetic peptide
GETRAPL

SEQ ID NO: 21 - protein, 7 aa, Artificial Sequence
  Description of Artificial Sequence Synthetic peptide
GNVLRTN

SEQ ID NO: 22 - protein, 7 aa, Artificial Sequence
  Description of Artificial Sequence Synthetic peptide
GTNPSLR

SEQ ID NO: 23 - protein, 7 aa, Artificial Sequence
  Description of Artificial Sequence Synthetic peptide
GTYPTFV

SEQ ID NO: 24 - protein, 7 aa, Artificial Sequence
  Description of Artificial Sequence Synthetic peptide
GYLNTLY

SEQ ID NO: 25 - protein, 7 aa, Artificial Sequence
  Description of Artificial Sequence Synthetic peptide
HAAFMYP

SEQ ID NO: 26 - protein, 7 aa, Artificial Sequence
  Description of Artificial Sequence Synthetic peptide
HANYPAW

SEQ ID NO: 27 - protein, 7 aa, Artificial Sequence
  Description of Artificial Sequence Synthetic peptide
HFRLLDQ

SEQ ID NO: 28 - protein, 7 aa, Artificial Sequence
  Description of Artificial Sequence Synthetic peptide
HNFPQVW

SEQ ID NO: 29 - protein, 7 aa, Artificial Sequence
  Description of Artificial Sequence Synthetic peptide
HPHHRIF

SEQ ID NO: 30 - protein, 7 aa, Artificial Sequence
  Description of Artificial Sequence Synthetic peptide
HRVYTTP

SEQ ID NO: 31 - protein, 7 aa, Artificial Sequence
  Description of Artificial Sequence Synthetic peptide
IFASVPS

SEQ ID NO: 32 - protein, 7 aa, Artificial Sequence
  Description of Artificial Sequence Synthetic peptide
IIVPRLG

SEQ ID NO: 33 - protein, 7 aa, Artificial Sequence
  Description of Artificial Sequence Synthetic peptide
IQSPHFI

SEQ ID NO: 34 - protein, 7 aa, Artificial Sequence
  Description of Artificial Sequence Synthetic peptide
KPAVYTK

SEQ ID NO: 35 - protein, 7 aa, Artificial Sequence
  Description of Artificial Sequence Synthetic peptide
KPPEFLL

SEQ ID NO: 36 - protein, 7 aa, Artificial Sequence
  Description of Artificial Sequence Synthetic peptide
KQTLPSA

SEQ ID NO: 37 - protein, 7 aa, Artificial Sequence
  Description of Artificial Sequence Synthetic peptide
KTPMTYD

SEQ ID NO: 38 - protein, 7 aa, Artificial Sequence
  Description of Artificial Sequence Synthetic peptide
MNPSPAA

SEQ ID NO: 39 - protein, 7 aa, Artificial Sequence
  Description of Artificial Sequence Synthetic peptide
MPIPRPP

SEQ ID NO: 40 - protein, 7 aa, Artificial Sequence
  Description of Artificial Sequence Synthetic peptide
MPSPDIY

SEQ ID NO: 41 - protein, 7 aa, Artificial Sequence
  Description of Artificial Sequence Synthetic peptide
QLFTSAS

SEQ ID NO: 42 - protein, 7 aa, Artificial Sequence
  Description of Artificial Sequence Synthetic peptide
QMPLPDT

SEQ ID NO: 43 - protein, 7 aa, Artificial Sequence
  Description of Artificial Sequence Synthetic peptide
QPPHPYR

SEQ ID NO: 44 - protein, 7 aa, Artificial Sequence
  Description of Artificial Sequence Synthetic peptide
QPTQLTR

SEQ ID NO: 45 - protein, 7 aa, Artificial Sequence
  Description of Artificial Sequence Synthetic peptide
QQFPQST

SEQ ID NO: 46 - protein, 7 aa, Artificial Sequence
  Description of Artificial Sequence Synthetic peptide
QSPPVGH

SEQ ID NO: 47 - protein, 7 aa, Artificial Sequence
  Description of Artificial Sequence Synthetic peptide
RAPKFLN

SEQ ID NO: 48 - protein, 7 aa, Artificial Sequence
  Description of Artificial Sequence Synthetic peptide
SALFHNK

SEQ ID NO: 49 - protein, 7 aa, Artificial Sequence
  Description of Artificial Sequence Synthetic peptide
SDPPISW

SEQ ID NO: 50 - protein, 7 aa, Artificial Sequence
  Description of Artificial Sequence Synthetic peptide
SEGRLYK

SEQ ID NO: 51 - protein, 7 aa, Artificial Sequence
  Description of Artificial Sequence Synthetic peptide
SLLDLIH

SEQ ID NO: 52 - protein, 7 aa, Artificial Sequence
  Description of Artificial Sequence Synthetic peptide
SNQLLMT

SEQ ID NO: 53 - protein, 7 aa, Artificial Sequence
  Description of Artificial Sequence Synthetic peptide
SQNFGFS

SEQ ID NO: 54 - protein, 7 aa, Artificial Sequence
  Description of Artificial Sequence Synthetic peptide
SWSNVVV

SEQ ID NO: 55 - protein, 7 aa, Artificial Sequence
  Description of Artificial Sequence Synthetic peptide
THQARSV

SEQ ID NO: 56 - protein, 7 aa, Artificial Sequence
  Description of Artificial Sequence Synthetic peptide
MEGYIHR

SEQ ID NO: 57 - protein, 7 aa, Artificial Sequence
  Description of Artificial Sequence Synthetic peptide
PTPMTLY

SEQ ID NO: 58 - protein, 7 aa, Artificial Sequence
  Description of Artificial Sequence Synthetic peptide
HAIYPRH

SEQ ID NO: 59 - protein, 7 aa, Artificial Sequence
  Description of Artificial Sequence Synthetic peptide
SEGRLYK

SEQ ID NO: 60 - protein, 7 aa, Artificial Sequence
  Description of Artificial Sequence Synthetic peptide
ALQYHNL

SEQ ID NO: 61 - protein, 7 aa, Artificial Sequence
  Description of Artificial Sequence Synthetic peptide
THQARSV

SEQ ID NO: 62 - protein, 7 aa, Artificial Sequence
  Description of Artificial Sequence Synthetic peptide
SNQLLMT

SEQ ID NO: 63 - protein, 7 aa, Artificial Sequence
  Description of Artificial Sequence Synthetic peptide
PPRPIPM

SEQ ID NO: 64 - protein, 7 aa, Artificial Sequence
  Description of Artificial Sequence Synthetic peptide
HPHHRIF

SEQ ID NO: 65 - protein, 7 aa, Artificial Sequence
  Description of Artificial Sequence Synthetic peptide
MPIPRPP

SEQ ID NO: 66 - protein, 7 aa, Artificial Sequence
  Description of Artificial Sequence Synthetic peptide
STTKLAL

SEQ ID NO: 67 - DNA, 21 bp, Artificial Sequence
  Description of Artificial Sequence Synthetic oligonucleotide
agtagtaagctgaggaacagt

SEQ ID NO: 68 - DNA, 21 bp, Artificial Sequence
  Description of Artificial Sequence Synthetic oligonucleotide
caaagccaacttagtcgtcga

SEQ ID NO: 69 - DNA, 21 bp, Artificial Sequence
  Description of Artificial Sequence Synthetic oligonucleotide
ccgatgaatataaccctccat

SEQ ID NO: 70 - DNA, 21 bp, Artificial Sequence
  Description of Artificial Sequence Synthetic oligonucleotide
cggaagcggagtcaacggcag

SEQ ID NO: 71 - DNA, 21 bp, Artificial Sequence
  Description of Artificial Sequence Synthetic oligonucleotide
atgacgcggataaatagcatg

SEQ ID NO: 72 - DNA, 21 bp, Artificial Sequence
  Description of Artificial Sequence Synthetic oligonucleotide
ctgatcaagcagcctagaatg

SEQ ID NO: 73 - DNA, 21 bp, Artificial Sequence
  Description of Artificial Sequence Synthetic oligonucleotide
aaaaaaatgaggagactgaat

SEQ ID NO: 74 - DNA, 21 bp, Artificial Sequence
  Description of Artificial Sequence Synthetic oligonucleotide
atgagaacgatgcgcagcata

SEQ ID NO: 75 - DNA, 21 bp, Artificial Sequence
  Description of Artificial Sequence Synthetic oligonucleotide
cggcgtacgcatcctatccgc

SEQ ID NO: 76 - DNA, 21 bp, Artificial Sequence
  Description of Artificial Sequence Synthetic oligonucleotide
attatgagaaaccggaaaagc

SEQ ID NO: 77 - DNA, 21 bp, Artificial Sequence
  Description of Artificial Sequence Synthetic oligonucleotide
ccacggccaatcatcatgagc

SEQ ID NO: 78 - DNA, 21 bp, Artificial Sequence
  Description of Artificial Sequence Synthetic oligonucleotide
catactatgatgcggatgcgc

SEQ ID NO: 79 - DNA, 21 bp, Artificial Sequence
  Description of Artificial Sequence Synthetic oligonucleotide
cagattatgatactgaagagc

SEQ ID NO: 80 - DNA, 21 bp, Artificial Sequence
  Description of Artificial Sequence Synthetic oligonucleotide
cgaccgatgaggagaagacgc

SEQ ID NO: 81 - DNA, 21 bp, Artificial Sequence
  Description of Artificial Sequence Synthetic oligonucleotide
ccgatccgacggattccaagc

SEQ ID NO: 82 - DNA, 21 bp, Artificial Sequence
  Description of Artificial Sequence Synthetic oligonucleotide
acgatacaccggagtattatc

SEQ ID NO: 83 - DNA, 21 bp, Artificial Sequence
  Description of Artificial Sequence Synthetic oligonucleotide
catcttcagctgcagcggctc

SEQ ID NO: 84 - DNA, 21 bp, Artificial Sequence
  Description of Artificial Sequence Synthetic oligonucleotide
atgctgctgagacgaaggaaa

SEQ ID NO: 85 - DNA, 21 bp, Artificial Sequence
  Description of Artificial Sequence Synthetic oligonucleotide
cagatgcgcatgagccgaaaa

SEQ ID NO: 86 - DNA, 21 bp, Artificial Sequence
  Description of Artificial Sequence Synthetic oligonucleotide
aagcggcgcacgagtctcccc

SEQ ID NO: 87 - DNA, 21 bp, Artificial Sequence
  Description of Artificial Sequence Synthetic oligonucleotide
attcgtcctcaaaacattccc

SEQ ID NO: 88 - DNA, 21 bp, Artificial Sequence
  Description of Artificial Sequence Synthetic oligonucleotide
acgcagcgacggattagtacc

SEQ ID NO: 89 - DNA, 21 bp, Artificial Sequence
  Description of Artificial Sequence Synthetic oligonucleotide
aacaaaagtaggatacgtccc

SEQ ID NO: 90 - DNA, 21 bp, Artificial Sequence
  Description of Artificial Sequence Synthetic oligonucleotide
atacaaagtattaagataacc

SEQ ID NO: 91 - DNA, 21 bp, Artificial Sequence
  Description of Artificial Sequence Synthetic oligonucleotide
aggatacataaaagccgcatg

SEQ ID NO: 92 - DNA, 21 bp, Artificial Sequence
  Description of Artificial Sequence Synthetic oligonucleotide
ccaagcaggataattagcatg

SEQ ID NO: 93 - DNA, 21 bp, Artificial Sequence
  Description of Artificial Sequence Synthetic oligonucleotide
ctgatcaagcagcctagaatg

SEQ ID NO: 94 - DNA, 21 bp, Artificial Sequence
  Description of Artificial Sequence Synthetic oligonucleotide
ccacacctgcggaaaattatg

SEQ ID NO: 95 - DNA, 21 bp, Artificial Sequence
  Description of Artificial Sequence Synthetic oligonucleotide
aaaaatccgatgatgcggatg

SEQ ID NO: 96 - DNA, 21 bp, Artificial Sequence
  Description of Artificial Sequence Synthetic oligonucleotide
aggcgtcgtataaaccctatg

SEQ ID NO: 97 - DNA, 21 bp, Artificial Sequence
  Description of Artificial Sequence Synthetic oligonucleotide
agaaggcaccgacgcaaaaat

SEQ ID NO: 98 - DNA, 21 bp, Artificial Sequence
  Description of Artificial Sequence Synthetic oligonucleotide
acccagcctcggaacaataat

SEQ ID NO: 99 - DNA, 21 bp, Artificial Sequence
  Description of Artificial Sequence Synthetic oligonucleotide
aaaaaaatgaggagactgaat

SEQ ID NO: 100 - DNA, 21 bp, Artificial Sequence
  Description of Artificial Sequence Synthetic oligonucleotide
cttcgtatacaccgccggctt

SEQ ID NO: 101 - DNA, 21 bp, Artificial Sequence
  Description of Artificial Sequence Synthetic oligonucleotide
aagcagaaactccggaggctt

SEQ ID NO: 102 - DNA, 21 bp, Artificial Sequence
  Description of Artificial Sequence Synthetic oligonucleotide
cgccgacggaagagtctgctt

SEQ ID NO: 103 - DNA, 21 bp, Artificial Sequence
  Description of Artificial Sequence Synthetic oligonucleotide
atcataagtcatcggcgtctt

SEQ ID NO: 104 - DNA, 21 bp, Artificial Sequence
  Description of Artificial Sequence Synthetic oligonucleotide
agcagcaggagacggattcat

SEQ ID NO: 105 - DNA, 21 bp, Artificial Sequence
  Description of Artificial Sequence Synthetic oligonucleotide
aggcggcctaggaataggcat

SEQ ID NO: 106 - DNA, 21 bp, Artificial Sequence
  Description of Artificial Sequence Synthetic oligonucleotide
ataaatatcaggactaggcat

SEQ ID NO: 107 - DNA, 21 bp, Artificial Sequence
  Description of Artificial Sequence Synthetic oligonucleotide
cgacgcagacgtaaacagctg

SEQ ID NO: 108 - DNA, 21 bp, Artificial Sequence
  Description of Artificial Sequence Synthetic oligonucleotide
agtatcaggaagaggcatctg

SEQ ID NO: 109 - DNA, 21 bp, Artificial Sequence
  Description of Artificial Sequence Synthetic oligonucleotide
ccgataaggatgaggaggctg

SEQ ID NO: 110 - DNA, 21 bp, Artificial Sequence
  Description of Artificial Sequence Synthetic oligonucleotide
ccgagtaagctgagtaggctg

SEQ ID NO: 111 - DNA, 21 bp, Artificial Sequence
  Description of Artificial Sequence Synthetic oligonucleotide
cgtagactgcggaaactgctg

SEQ ID NO: 112 - DNA, 21 bp, Artificial Sequence
  Description of Artificial Sequence Synthetic oligonucleotide
atgcccaacaggcggcgactg

SEQ ID NO: 113 - DNA, 21 bp, Artificial Sequence
  Description of Artificial Sequence Synthetic oligonucleotide
attaagaaacttcggagccag

SEQ ID NO: 114 - DNA, 21 bp, Artificial Sequence
  Description of Artificial Sequence Synthetic oligonucleotide
cttctgatgataaagagcact

SEQ ID NO: 115 - DNA, 21 bp, Artificial Sequence
  Description of Artificial Sequence Synthetic oligonucleotide
ccaagaaatcggcggatcaga

SEQ ID NO: 116 - DNA, 21 bp, Artificial Sequence
  Description of Artificial Sequence Synthetic oligonucleotide
cttataaagacgcccctcaga

SEQ ID NO: 117 - DNA, 21 bp, Artificial Sequence
  Description of Artificial Sequence Synthetic oligonucleotide
atgaataagatcaagcaaact

SEQ ID NO: 118 - DNA, 21 bp, Artificial Sequence
  Description of Artificial Sequence Synthetic oligonucleotide
cgtcataagcagctgattcga

SEQ ID NO: 119 - DNA, 21 bp, Artificial Sequence
  Description of Artificial Sequence Synthetic oligonucleotide
cgaaaaaccaaaattctgcga

SEQ ID NO: 120 - DNA, 21 bp, Artificial Sequence
  Description of Artificial Sequence Synthetic oligonucleotide
cacaaccacattcgaccacga

SEQ ID NO: 121 - DNA, 21 bp, Artificial Sequence
  Description of Artificial Sequence Synthetic oligonucleotide
aaccgaacgcgcctgatgagt

SEQ ID NO: 122 - DNA, 18 bp, Artificial Sequence
  Description of Artificial Sequence Synthetic primer
ccctcatagttagcgtaa

SEQ ID NO: 123 - DNA, 19 bp, Artificial Sequence
  Description of Artificial Sequence Synthetic primer
cgcgggattgtgactgact

SEQ ID NO: 124 - DNA, 20 bp, Artificial Sequence
  Description of Artificial Sequence Synthetic primer
gccaaaagagccgtcaactt

SEQ ID NO: 125 - DNA, 24 bp, Artificial Sequence
  Description of Artificial Sequence Synthetic probe
agcagtgcagcttcccgttcatcc

SEQ ID NO: 126 - protein, 6 aa, Artificial Sequence
  Description of Artificial Sequence Synthetic peptide
EGYIHR

SEQ ID NO: 127 - DNA, 20 bp, Artificial Sequence
  Description of Artificial Sequence Synthetic primer
ccctcatagttagcgtaacg